(19) United States
(12) Patent Application Publication
(10) Pub. No.: US 2022/0317132 A1
(43) Pub. Date: Oct. 6, 2022

(54) USE OF ENDOTHELIN RECEPTOR INHIBITOR FOR INHIBITING EXOSOME SECRETION OR INHIBITING PD-L1 EXPRESSION

(71) Applicant: KYUNGPOOK NATIONAL UNIVERSITY INDUSTRY-ACADEMIC COOPERATION FOUNDATION, Seoul (KR)

(72) Inventors: Moon-Chang BAEK, Daegu (KR); Eun-Ju IM, Daegu (KR); Chan-Hyeong LEE, Daegu (KR)

(73) Assignee: KYUNGPOOK NATIONAL UNIVERSITY INDUSTRY-ACADEMIC COOPERATION FOUNDATION, Seoul (KR)

(21) Appl. No.: 17/642,250

(22) PCT Filed: Sep. 9, 2020
(86) PCT No.: PCT/KR2020/012189

§ 371 Date: Mar. 11, 2022

(30) Foreign Application Priority Data
Sep. 11, 2019 (KR) 10-2019-0112696

(51) Int. Cl. G01N 33/68; G01N 33/50

(52) U.S. Cl. CPC G01N 33/6872; G01N 33/5011

(57) ABSTRACT

The present invention relates to a use of an endothelin receptor inhibitor for inhibiting exosome secretion or inhibiting PD-L1 expression. It was confirmed that the endothelin receptor inhibitor inhibits endothelin receptors, which are currently known as a target for anticancer drug development, to not only inhibit the secretion of cancer cell-derived exosomes, but also reduce the expression of PD-L1 in cells, and is thus effective for cancer treatment when used in combination with existing anticancer drugs or when used alone in the form of a drug. Accordingly, the present invention can be used in novel modes of anticancer drug development using drugs exhibiting immune-checkpoint inhibition effects and exosome secretion inhibition effects.

### TECHNICAL FIELD

The present invention relates to a use of an endothelin receptor inhibitor for inhibiting exosome secretion or inhibiting programmed cell death-ligand 1 (PD-L1) expression.

### BACKGROUND ART

Exosomes are small membrane vesicles secreted from most cells. The diameter of exosomes is approximately 30-100 nm, and exosomes contain various types of proteins, genetic materials (DNA, RNA, miRNA), lipids, and the like, originating from the cell. Exosomes originate in specific intracellular compartments called multivesicular bodies (MVBs) and are released and secreted out of the cell, rather than directly detaching from plasma membrane. In other words, when fusion of the multivesicular bodies and the plasma membrane occurs, vesicles are released into the extracellular environment, which are called exosomes. Although it has not been identified exactly by what mechanism exosomes are made, it is known that they are isolated and released from various cell types under both normal and pathological conditions.

In addition, programmed cell death (PD-1), one of immune-checkpoint molecules, has become an attractive therapeutic target in several cancers. PD-1 is upregulated in T cells upon activation and is highly present in depleted T cells commonly seen in tumor-infiltrating lymphocytes. When interaction between PD-1 and its ligand, PD-L1, was blocked, excellent antitumor response and clinical effects were confirmed in some patients.

Recently, research and development of various cancer treatment methods based on immunotherapy are in full swing. In addition, it has been revealed that immunotherapeutic agents approved by FDA have many advantages for treatment of various cancers. However, due to a high cost of treatment and an issue that not all patients respond in common, they have the disadvantage that they may be used only for specific patients. Accordingly, it is necessary to research and develop novel modes of anticancer drug using drugs exhibiting immune-checkpoint inhibition effects and exosome secretion inhibition effects.

### DISCLOSURE

### Technical Problem

An object of the present invention is to provide an exosome secretion inhibitor or programmed cell death-ligand 1 (PD-L1) expression inhibitor screening method comprising selecting a test substance in which level of activity of an endothelin receptor is inhibited.

In addition, another object of the present invention is to provide a reagent composition for inhibiting exosome secretion or inhibiting PD-L1 expression of cancer cells in vitro, comprising an endothelin receptor inhibitor as an active ingredient.

In addition, still another object of the present invention is to provide a method of inhibiting exosome secretion or inhibiting PD-L1 expression in cancer cells, comprising inhibiting endothelin receptor activity of cancer cells in vitro.

### Technical Solution

An aspect of the present invention provides an exosome secretion inhibitor or programmed cell death-ligand 1 (PD-L1) expression inhibitor screening method comprising (1) contacting a test substance with cancer cells; (2) measuring level of activity of an endothelin receptor in the cancer cells contacted with the test substance; and (3) selecting a test substance in which the level of activity of the endothelin receptor is inhibited as compared to a control sample.

Further, an aspect of the present invention provides a reagent composition for inhibiting exosome secretion or inhibiting PD-L1 expression of cancer cells in vitro, comprising an endothelin receptor inhibitor as an active ingredient.

Furthermore, an aspect of the present invention provides a method of inhibiting exosome secretion or inhibiting PD-L1 expression in cancer cells, comprising inhibiting endothelin receptor activity of cancer cells in vitro.

### Advantageous Effects

The present invention relates to a use of an endothelin receptor inhibitor for inhibiting exosome secretion or inhibiting PD-L1 expression. It was confirmed that the endothelin receptor inhibitor inhibits endothelin receptors, which are currently known as a target for anticancer drug development, to not only inhibit the secretion of cancer cell-derived exosomes, but also reduce the expression of PD-L1 in cells, and is thus effective for cancer treatment when used in combination with existing anticancer drugs or when used alone in the form of a drug. Accordingly, the present invention may be used in novel modes of anticancer drug development using drugs exhibiting immune-checkpoint inhibition effects and exosome secretion inhibition effects.

### BRIEF DESCRIPTIONS OF THE DRAWINGS

FIG. 1 is a diagram illustrating results of identifying cytotoxic effects of endothelin receptor inhibitors.

FIG. 2 is a diagram illustrating results of identifying expression level of PD-L1 in cells and exosomes of MCF10A, which is normal breast cell, and MCF7 and MDA-MB231, which are breast cancer cells.

FIG. 3 is a diagram illustrating results of identifying PD-L1 expressing exosome secretion inhibition effects of endothelin receptor inhibitors.

FIG. 4 is a diagram illustrating results of identifying PD-L1 mRNA expression inhibition effects in cells of endothelin receptor inhibitors.

FIG. 5 is a diagram illustrating results of identifying PD-L1 protein expression inhibition effects in cells of endothelin receptor inhibitors.

FIG. 6 is a diagram illustrating results of identifying PD-L1 expressing exosome secretion inhibition effects in vivo of endothelin receptor inhibitors.

### BEST MODE

The present invention provides an exosome secretion inhibitor or programmed cell death-ligand 1 (PD-L1) expression inhibitor screening method comprising, (1) contacting a test substance with cancer cells, (2) measuring level of activity of an endothelin receptor in cancer cells contacted with the test substance, and (3) selecting a test substance in which the level of activity of the endothelin receptor is inhibited as compared to a control sample.

Specifically, the PD-L1 expression inhibitor may inhibit PD-L1 expression in the cancer cells or expression of exosome surface PD-L1 secreted from the cancer cells, but is not limited thereto.

Preferably, the cancer cells may be breast cancer cells, but are not limited thereto.

The term “test substance” used while referring to the screening method of the present invention refers to an unknown candidate substance used in screening to test whether or not it affects expression level of a gene or affects expression or activity of a protein, or affects binding between proteins. The sample includes chemicals, nucleotides, antisense-RNA, small interference RNA (siRNA), and natural product extracts, but is not limited thereto.

In addition, the present invention provides a reagent composition for inhibiting exosome secretion or inhibiting PD-L1 expression of cancer cells in vitro, comprising an endothelin receptor inhibitor as an active ingredient.

Specifically, the endothelin receptor inhibitor may be any one or more selected from the group consisting of sulfisoxazole (SFX), ambrisentan, macitentan, and bosentan, but is not limited thereto.

Specifically, the PD-L1 expression inhibitor may inhibit the PD-L1 expression in the cancer cells or the expression of exosome surface PD-L1 secreted from cancer cells, but is not limited thereto.

Preferably, the cancer cells may be breast cancer cells, but are not limited thereto.

In addition, the present invention provides a method of inhibiting exosome secretion or inhibiting PD-L1 expression in cancer cells, comprising inhibiting endothelin receptor activity of cancer cells in vitro.

Specifically, the endothelin receptor inhibitor may be any one or more selected from the group consisting of sulfisoxazole (SFX), ambrisentan, macitentan and bosentan, but is not limited thereto.

Specifically, the PD-L1 expression inhibitor may inhibit the PD-L1 expression in the cancer cells or the expression of exosome surface PD-L1 secreted from the cancer cells, but is not limited thereto.

Preferably, the cancer cells may be breast cancer cells, but are not limited thereto.

### MODES FOR CARRYING OUT INVENTION

Hereinafter, examples will be described in detail to help understanding of the present disclosure. However, the examples below are merely illustrative of content of the invention, and the scope of the invention is not limited to the examples below. The examples of the disclosure are provided to more completely explain the invention to a person skilled in the art.

### Experimental Example

The experimental examples below are intended to provide experimental examples commonly applied to each example according to the present invention.

1. Preparation of Sample

Sulfisoxazole was purchased from sigma (31739). Ambrisentan was purchased from Medchem Express (HY13209), bosentan was purchased from Medchem Express (HY-A0013), and macitentan was purchased from Medchem Express (HY-14184). For cell experiments, they were dissolved in DMSO (Dimetyl sulfoxide) and used.

2. Cell Culture Conditions

MCF10A cells were cultured in Mammary epithelial cell growth medium (MEGM, Lonza) added with 5% fetal bovine serum, 1% antibiotics, 52 μg/ml bovine pituitary extract, 0.5 μg/ml hydrocortisone, 10 ng/ml EGF, and 5 μg/ml insulin. MDA-MB231 and MCF7 cells were cultured in Dulbecco's modified Eagle's medium (DMEM, Hyclone) added with 10% fetal bovine serum and 1% antibiotics.

### <Example 1> Confirmation of Cytotoxic Effect (MTT Analysis)

MDA-MB231 cells were seeded at 1×10⁵/well in a 24-well plate, and then cultured for 24 hours to allow time for cell stabilization. After culturing for 24 hours, the cells were treated with 100 μM of sulfisoxazole, 1, 10, and 100 μM of ambrisentan, 100 and 1000 nM of macitentan, and 10 and 50 μM of bosentan and cultured for 24 hours. Then, after treatment with MTT tetrazolium reagent, the cells were cultured for 4 hours. After 4 hours, reduced MTT formazan (3-(4,5-dimethylthiazol-2-yl)-2,5 diphenyl-tetrazolium bromide) was measured at 595 nm absorbance to confirm cell proliferation effects.

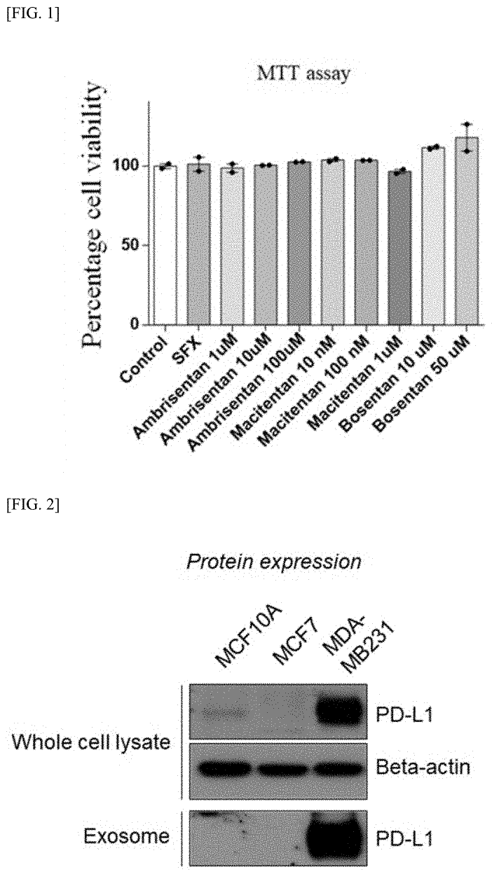

Results for this was illustrated in FIG. 1.

As illustrated in FIG. 1, it was confirmed that sulfisoxazole (SFX), ambrisentan, macitentan, and bosentan exhibited no cytotoxicity at concentrations used.

### <Example 2> Confirmation of PD-L1 Expression in Cells and Exosomes

After culturing MCF10A, which is normal breast cells, and MCF7 and MDA-MB231, which are breast cancer cells, secreted exosomes were separated using ultracentrifugation (300×g/3 min, 2,500×g/15 min, 10,000×g/30 min, 120,000×g/90 min). To identify expression level of PD-L1 in cells and exosomes, proteins were extracted from the cells and exosomes using 1× RIPA buffer. The extracted proteins were reacted with PD-L1 antibody through western blotting, and proteins expression level was measured using ECL. Beta-actin was used as a loading control.

Results for this were illustrated in FIG. 2.

As illustrated in FIG. 2, it was confirmed that PD-L1 was strongly expressed in exosomes derived from MDA-MB231 cells and MDA-MB231 cells.

### <Example 3> PD-L1 Expressing Exosome Secretion Inhibition Effect

MDA-MB231 cells, a breast cancer cell line, were cultured in a 24-well plate. After culturing for 24 hours, medium was removed, cells were washed with PBS, and were treated with drug with a serum-free medium not including phenol-red. Control group was treated with DMSO. After culturing for 24 hours and removing the medium, they were centrifuged at 300×g/3 min, 2,500×g/15 min, and 10,000×g/30 min, and supernatant was transferred to anew tube. The number of exosomes secreted from the cells were measured using Nano-sight LM10 (Malvern) machine.

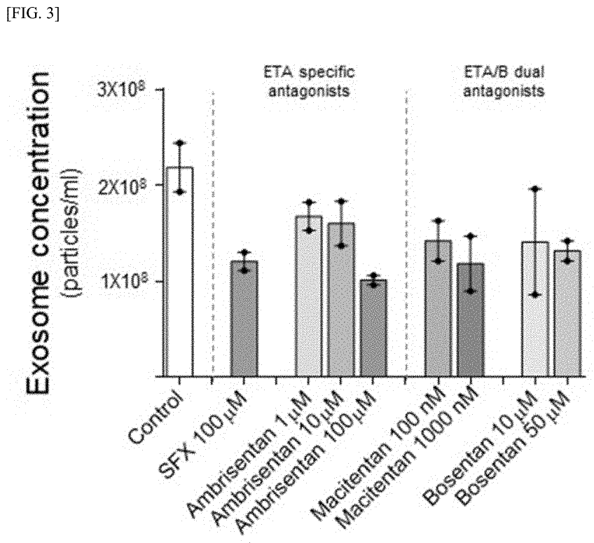

Results for this were illustrated in FIG. 3.

As illustrated in FIG. 3, both ETA specific antagonist and ETA/B dual antagonist effectively inhibited PD-L1 expressed MDA-MB231 cells-derived exosomes secretion.

### <Example 4> PD-L1 mRNA Expression Inhibition Effect in Cell

MDA-MB231 cells, a breast cancer cell line, were cultured in a 6 well plate. After culturing for 24 hours, medium was removed, and cells were washed with PBS and treated with drug with a serum-free medium. Control group was treated with DMSO. After culturing for 24 hours, total RNA was isolated using Trizol. After synthesizing with cDNA through RT-PCR, PD-L1 mRNA was measured using quantitative PCR.

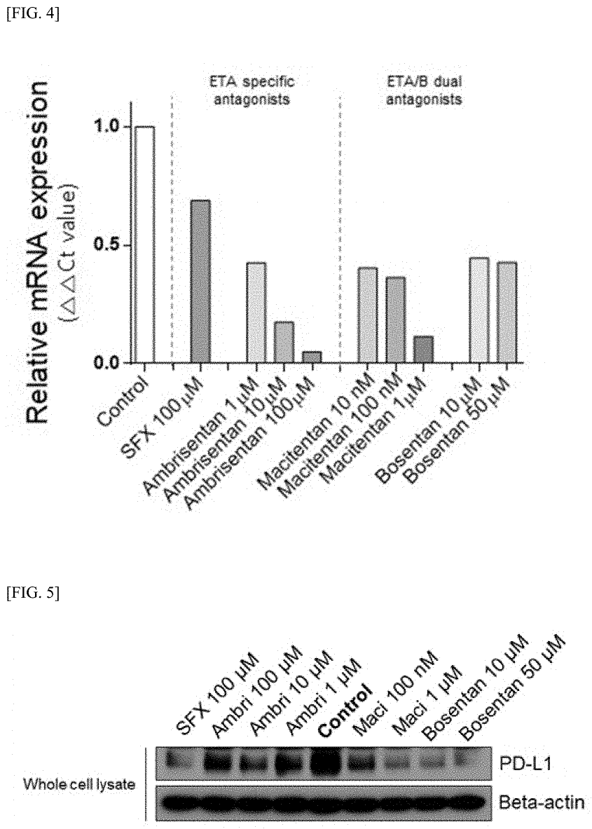

Results for this were illustrated in FIG. 4.

As illustrated in FIG. 4, both ETA specific antagonist and ETA/B dual antagonist effectively inhibited mRNA expression change of PD-L1 in MDA-MB231 cells.

### <Example 5> PD-L1 Protein Expression Inhibition Effect in Cell

MDA-MB231 cells, a breast cancer cell line, were cultured in a 6 well plate. After culturing for 24 hours, medium was removed, and cells were washed with PBS and treated with the drugs with a serum-free medium. Control group was treated with DMSO. After culturing for 24 hours, total protein was isolated using 1×RIPA buffer. Proteins were reacted with PD-L1 antibody through western blotting, and expression level was measured using ECL.

Results for this were illustrated in FIG. 5.

As illustrated in FIG. 5, both the ETA specific antagonist and the ETA/B dual antagonist strongly inhibited PD-L1 protein expression change in MDA-MB231 cells.

### <Example 6> PD-L1 Expressing Exosome Secretion Inhibition Effect In Vivo

A cell line that increased or inhibited expression of ETA and ETB in human breast cancer cells (MDA-MB231) (control; shCtrl, ETA knock down; ETA K/D, ETB Knock down; ETB K/D, ETA over-expression; ETA 0/E, ETB over-expression; ETB 0/E) was orthotopic transplanted into Balb/c nude female mice, and 14 days later, blood was collected and plasma was isolated using an EDTA tube. Exosomes in plasma were separated using ultracentrifugation (300×g/3 min, 2,500×g/15 min, 10,000×g/30 min, 160,000×g/120 min). To measure PD-L1 expression breast cancer cell-derived exosomes in the whole exosomes, total protein was isolated from the exosomes and reacted with PD-L1 antibody through western blotting. Expression level was measured using ECL.

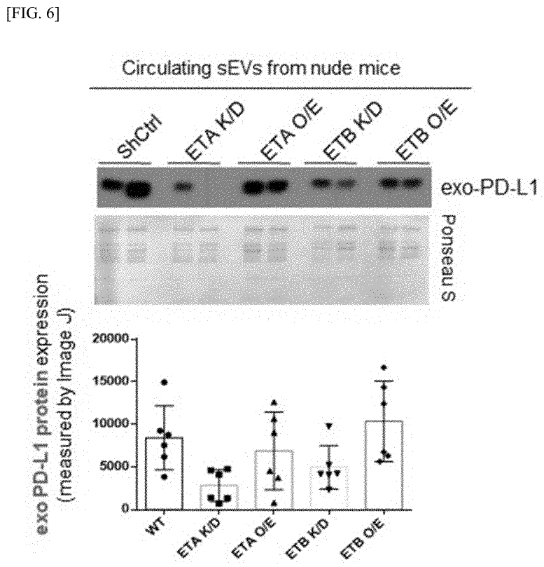

Results for this were illustrated in FIG. 6.

As illustrated in FIG. 6, when the expression of ETA and ETB was increased, there was no significant change, but when the expression of ETA and ETB was decreased, it was observed that the amount of PD-L1 expressing exosomes decreased.

1. An exosome secretion inhibitor or programmed cell death-ligand 1 (PD-L1) expression inhibitor screening method, the screening method comprising:
(1) contacting a test substance with cancer cells;
(2) measuring level of activity of an endothelin receptor in the cancer cells contacted with the test substance; and
(3) selecting a test substance in which the level of activity of the endothelin receptor is inhibited as compared to a control sample.

2. The screening method of claim 1, wherein the PD-L1 expression inhibitor inhibits PD-L1 expression in the cancer cells or expression of exosome surface PD-L1 secreted from the cancer cells.

3. The screening method of claim 1, wherein the cancer cells are breast cancer cells.

4. A reagent composition for inhibiting exosome secretion or inhibiting PD-L1 expression of cancer cells in vitro, comprising an endothelin receptor inhibitor as an active ingredient.

5. The reagent composition of claim 4, wherein the endothelin receptor inhibitor is any one or more selected from the group consisting of sulfisoxazole (SFX), ambrisentan, macitentan, and bosentan.

6. The reagent composition of claim 4, wherein the inhibiting of the PD-L1 expression comprises inhibiting PD-L1 expression in the cancer cells or expression of exosome surface PD-L1 secreted from the cancer cells.

7. The reagent composition of claim 4, wherein the cancer cells are breast cancer cells.

8. A method of inhibiting exosome secretion or inhibiting PD-L1 expression in cancer cells, the method comprising inhibiting endothelin receptor activity of cancer cells in vitro.

9. The method of claim 8, wherein the inhibiting of the endothelin receptor activity comprises treating with any one or more selected from the group consisting of sulfisoxazole (SFX), ambrisentan, macitentan, and bosentan.

10. The method of claim 8, wherein the inhibiting of the PD-L1 expression comprises inhibiting PD-L1 expression in the cancer cells or expression of exosome surface PD-L1 secreted from the cancer cells.

11. The method of claim 8, wherein the cancer cells are breast cancer cells.
